(19) United States
(12) Patent Application Publication
(10) Pub. No.: US 2017/0224824 A1
(43) Pub. Date: Aug. 10, 2017

(54) WARMING PERSONAL LUBRICANT FREE FROM PRESERVATIVES

(71) Applicant: Guy & O'Neill, Inc., Fredonia, WI (US)

(72) Inventors: Wei Chen, Cedarburg, WI (US); Zachary RB McGinn, Hubertus, WI (US); Adam J. Ewald, Saukville, WI (US); Mahesh M. Bhide, Belgium, WI (US); Tarry P. Zielinski, Sussex, WI (US); Thomas C. Misgen, Random Lake, WI (US)

(21) Appl. No.: 15/423,733

(22) Filed: Feb. 3, 2017

Related U.S. Application Data
(60) Provisional application No. 62/291,901, filed on Feb. 5, 2016.

(51) Int. Cl. A61K 47/10; A61K 9/00

(52) U.S. Cl. CPC A61K 47/10; A61K 9/0034

(57) ABSTRACT

Disclosed herein is a composition comprised of glycerin and 1,3-propanediol. The compositions can be used as a personal care lubricant, for drug delivery, or as a moisturizer, among other uses.

### CROSS-REFERENCE TO RELATED APPLICATIONS

This application claims priority to U.S. Provisional Application No. 62/291,901, filed Feb. 5, 2016, and is herein incorporated by reference in its entirety.

### BACKGROUND

The present invention relates to the field of personal lubricants, which have been shown to be beneficial for a number of clinical and personal applications. Attempts have been made to increase the shelf-life of these compositions, and often a preservative is included for this purpose. Preservatives known in the art, such as parabens, phenoxyethanol, and formaldehyde have been linked to health concerns, and can mediate unintended health consequences when included in a personal lubricant. The compositions of this invention are substantially free of preservatives while retaining the ability to act as a warming lubricant when applied to a mucosal surface or the skin. What is needed in the art of human personal lubricants is a preservative-free composition that can be applied without the known side-effects of preservatives known to those in the art.

### SUMMARY

In one aspect, disclosed is a personal care composition comprising glycerin and 1,3-propanediol. The invention can be used as a personal care lubricant, for drug delivery, and to provide warmth to tissues. Other aspects of the invention will become apparent by consideration of the detailed description.

### DETAILED DESCRIPTION

Disclosed herein is a composition comprised of glycerin and 1,3-propanediol. The compositions can be used as a personal care lubricant, for drug delivery, or as a moisturizer, among other uses.

### 1. Definitions

Unless otherwise defined, all technical and scientific terms used herein have the same meaning as commonly understood by one of ordinary skill in the art. In case of conflict, the present document, including definitions, will control. Suitable methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Composition

In one aspect, the invention provides a personal care composition comprising glycerin and 1,3-propanediol. In another aspect, the invention provides a personal care composition consisting essentially of glycerin and 1,3-propanediol. In yet another aspect, the invention provides a personal care composition consisting of glycerin and 1,3-propanediol.

In embodiments, the glycerin is present in an amount of about 5% to about 95%. In embodiments, the 1,3-propanediol is present in an amount of about 95% to about 5%.

The compositions of this invention are self-preserving and may not require a preservative. This is beneficial as preservatives known in the art have unintended consequences on health, and therefore omitting a preservative from these compositions is a unique characteristic of the invention. Thus, in an embodiment, the compositions may be substantially free of preservatives. By “substantially free,” it is meant that the compositions contains less than about 5% by weight of preservative, or less than about 3%, or less than about 1%, or less than about 0.5%, or less than about 0.3%, or less than about 0.1%, or less than about 0.01%.

Suitably, the invention is free of preservatives which include, but are not limited to, parabens, phenoxyethanol, and formaldehyde-donors. Parabens are synthetic preservatives, which can mimic the effect of estrogen in tissues, and have been linked with increasing the risk of breast cancer. Phenoxyethanol is a moderately hazardous chemical which has been linked to skin irritation and toxicity. High concentrations have been associated with reproductive damage, depressed immunity, and reduced nervous system function in newborns. Formaldehyde has been classified by the U.S government and the World Health Organization as carcinogenic when its fumes are inhaled. Additionally, formaldehyde is a potent skin sensitizer and allergen.

In embodiments, the compositions of the invention may also include mineral oil, plant extracts such as aloe, witch hazel, chamomile, hydrogenated soy oil and colloidal oatmeal, or vitamins such as vitamin A, D, or E.

In embodiments, water is present in an amount of about 0% to about 20%.

In embodiments, the compositions of this invention may further contain an insulating agent. Suitably, the insulating agent may be honey or esters of isopropyl alcohol and saturated high molecular weight fatty acids such as myristic or palmitic acid. The insulating agent may additionally be selected from the group consisting of, but not limited to, ethanol, niacin, jambu, nicotinic acid, zingerone, vanillyl alcohol isopropyl ether, gingerol, methyl salicylate, shogaol, paradol, zingerone, capsaicin, dihydrocapsaicin, nordihydrocapsaicin homocapsaicin, tincture capsicum, eucalyptus oil. The insulating agent may be present in the compositions of this invention in an amount of from about 1% to about 5% by weight of the composition. Suitably, the ester may include, but is not limited to, isopropyl stearate, isopropyl myristate, isopropyl palmitate, isopropyl laurate, and the like. Most suitably, the insulating agent is honey.

In embodiments, the compositions of this invention may contain one or more water-soluble natural or natural-derived polymers, such as cellulose-derived polymers, gums, chitosans or the like, which contribute to the viscosity and bioadhesiveness of the compositions of this invention. Suitably, such cellulose-derived polymers are hydroxyalkylcellulose polymers.

In embodiments, the compositions of this invention may also include flavoring agents to provide a sweet and pleasant taste, such as a sugar, which is of particular benefit when these compositions are used orally. Fragrances and aromas that enhance different senses and promote relaxation or intimacy may also be added to the compositions of this invention to enhance their effect, both in creating a feeling of relaxation and in improving intimacy. The desired fragrance may be obtained by including an essential oil, such as but not limited to, lavender, peppermint, tea tree oil, and eucalyptus. Colorants may be added to obtain a desirable colorimetric appearance.

In embodiments, the compositions of this invention may also include an extract, suitably a natural extract, such as green tea extract, rose hip extract, vanilla extract, almond extract, cherry extract, or aloe extract.

In embodiments, the compositions of this invention may be a liquid, a semi-solid, or a solid depending upon the particular intended use thereof. The compositions of this invention may also be formulated into soft or hard gelatin capsules, suppositories and impregnated into fabrics or polymers. The compositions of this invention may also be used in combination with barrier contraceptive methods, such as condoms.

The compositions of this invention may lubricate and soothe the tissues of the user, especially the oral and vaginal mucous membranes. The invention may also be used externally as a massage “oil”, or to lubricate the skin for easier application of a compressive sleeve, gloves or a condom. The compositions of this invention may also be used as moisturizers which convey a feeling of warmth and relieve dryness of tissues, such as vaginal dryness, dry mouth, dry and scaly skin, and to provide an ameliorating effect for frostbite on extremities over-exposed to the cold.

The compositions of this invention may provide a warming sensation. This warming effect has been found to enhance intimacy and increase pleasure during intimate activities, and may be useful for treating female sexual dysfunction by themselves as they may serve to increase blood flow to areas on which they are applied by increasing temperature thereon.

The compositions of this invention may also be used as a vehicle to deliver medication or other treatment agents to biomembranes including, but not limited to, antibiotics, or antiviral, antimicrobial or antifungal agents and the like. Suitable antibiotics may include, but are not limited to, metronidazole, clindamycin, tinidazole, ornidazole, seqnidazole, refaximin, trospectomycin, purpuromycin and their pharmaceutically acceptable salts and the like. Suitable antiviral agents may include, but are not limited to, immunomodulators such as imiquimod, its derivatives, podofilox, podophyllin, interferon alpha, reticolos, cidofovir, nonoxynol-9 and their pharmaceutically acceptable salts and the like. Suitable antimicrobial agents may include, but are not limited to, chlorohexidine gluconate, sodium polystyrene sulfonate, sodium cellulose sulfate, silver particles of micro- and sub-micrometer sizes, silver slats and other antibacterial agents known to the art. Suitable antifungal agent may include an azole or imidazole such as miconazole, econazole, terconazole, saperconazole, itraconazole, butaconazole, clotrimazole, tioconazole, fluconazole and ketoconazole, vericonazole, fenticonazole, sertaconazole, posaconazole, bifonazole, oxiconazole, sulconazole, elubiol, vorconazole, isoconazole, flutrimazole and their pharmaceutically acceptable salts and the like. Other antifungal agents may include but are limited to an allylamine or one from other chemical families such as ternafine, naftifine, amorolfine, butenafine, ciclopirox, griseofulvin, undecyclenic acid, haloprogin, tolnaftate, nystatin, iodine, rilopirox, BAY 108688, purpuromycin and their pharmaceutically acceptable salts.

The compositions of this invention may also include a solvent as a foamable carrier, including but not limited to, polyethylene glycols (PEGs) with or without a polypropylene glycol. Polyethylene glycol or derivatives or mixtures thereof and propylene glycol or derivatives are believed, in addition to their function as a solvent, to support, facilitate, improve or optimize the function and effect of active agents and may themselves have a therapeutic effect.

In one embodiment, the solvent is present at about 25% to about 98% by weight selected from the group consisting of (1) a polyethylene glycol (PEG) or derivative and (2) a propylene glycol or derivative or mixtures thereof.

The compositions of this invention may also include at least one polymeric agent, selected from the group consisting of a bioadhesive agent, a gelling agent, a film forming agent and a phase change agent. The polymeric agent is present in the composition in about 0.01% to about 5% by weight.

In one or more embodiments, the polymeric agent is present in the composition and is selected from the group consisting of locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose PEG 1000, PEG 4000, PEG 6000 and PEG 8000, polycarbophil, carbomer, ASOS, klucel and permulen.

The compositions of this invention may also include a humectant and/or an emollient to provide moisture to the mucosal surfaces to which they are applied.

In one embodiment, the humectant and/or emollient is selected from, but not limited to, alkylmonoglycerides, alkyldiglycerides, triglycerides, derivatives of these oils such as hydrogenated oils, diols such as 1,2-propanediol, 1,3-butanediol, cetyl alcohol, stearyl alcohol, oleic alcohol, glycol, butylene glycol, polyethylene glycol esters, polyethylene glycols or polypropylene glycols, fatty esters such as isopropyl palmitate, 2-ethylhexyl cocoate, myristyl myristate, isopropyl myristate, isopropyl stearate, glyceryl triacetate, ascorbyl palmitate, octyl dodecanol, hexyl decanol, oleyl alcohol, decyl oleate, hexyl laureate, dioctyl cyclohexane, glycearyl stearate, Ceteareth 20, Cetereath 12, cetyl palmitate, esters of lactic acid, stearic acid, behenic acid, isostearic acid, lecithin, collagen, elastin, keratin, lanolin, polydextrose, synthetic alcohols such as benzyl alcohol, butyl alcohol, cetearyl alcohol, cetyl alcohol, myristyl alcohol, diglyceryl caprylate and combinations thereof. In one embodiment, the humectant and/or emollient comprises from about or at 1% to about or at 20% of the composition. In another embodiment, the humectant and/or emollient comprises 1%, 2%, 3%, 4%, 5%, 6%, 7%, 8%, 9%, 10%, 11%, 12%, 13%, 14%, 15%, 16%, 17%, 18%, 19% or 20% of the composition.

The compositions of this invention may also include an anti-irritation agent, such as but not limited to, pyrithione or a polyvalent metal salt of pyrithione. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more suitably zinc. The zinc salt may be selected from the group consisting of, but not limited to: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof. Even more suitably for use herein is the zinc salt of 1-hydroxy-2-pyridinethione (known as “zinc pyrithione” or “ZPT”); more preferably ZPT in platelet particle form, wherein the particles have an average size of up to about 20 preferably up to about 5 more preferably up to about 2.5

In one embodiment, the anti-irritation agent comprises from about 0.01% to about 5% by weight; alternatively from about 0.05% to about 2%, alternatively from about 0.1% to about 1%, alternatively from about 0.2% to about 0.7%, alternatively about 0.5%. More suitably, an effective amount of a zinc salt has an aqueous solubility within the composition of less than about 25% by weight, at 25° C., more suitably less than about 20%; more suitably less than about 15%. Preferred embodiments of the present invention include from 0.001% to 10% of a zinc salt; more suitably from 0.01% to 5%; more suitably still from 0.1% to 3%. In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

The compositions of this invention may also include a “numbing” agent. “Numbing” is the feeling characterized by the loss of sensation in a particular area of the body. The numbing feeling may be utilized in compositions applied to a male partner in order to decrease sensitivity of the sexual organs. This can result in a prolongation of intimate physical activities. The local anesthetics may suitably include, but are not limited to, benzocaine, lidocaine, novocaine, tetracaine, dibucaine, lignocaine, mepivacaine, benzyl alcohol, camphor, resorcinol, menthol and diphenylhydramine hydrochloride and the like.

The compositions of this invention may also include a “flushing” agent. “Flushing” is the sensation created by enhanced blood flow in a body portion that can cause redness and engorgement of tissues. Engorgement of sexual organs in both females and males is an indication of arousal in both females and males. Certain natural products, such as pepper, chili pepper, ginger and the like may cause such a sensation.

The compositions of this invention may also include a “tingling” agent. “Tingling” is a unique sensation defined as “feeling a ringing, stinging, prickling or thrilling sensation” (Webster's Ninth New Collegiate Dictionary, Merriam-Webster, Springfield, Mass., 1987, p. 1236). Compositions that cause the tingling sensation can be used alone or with cooling or warming lubricant compositions to create a novel sensation. A tingling sensation may be imparted by some chemicals such as methyl salicylate, methyl nicotinate, menthyl lactate and other chemicals.

The compositions of the invention may include anionic, cationic, non-ionic or amphoteric surfactants to act as a wetting agent or foaming agent. Typical anionic surfactants are carboxylates, sulfonates, sulfates or phosphates, e.g. fatty acid soaps, salts of lauryl sulfate and salts of lauryl ether sulfate. Examples of cationic surfactants are aliphatic mono, di and polyamines derived from fatty and rosin acids, amine oxides, ethoxylated alkyl amines and imidazolines. Examples of non-ionic surfactants are polyoxyethylene surfactants, alkylphenol ethoxylates, carboxylic acid esters, e.g., mono and diglycerides, polyoxyethylene esters and fatty acid diethanolamine condensates. Amphoteric surfactants are those containing combinations of the anionic and cationic groups described above, particularly those containing both acid carboxyls and basic nitrogen groups. Typical amphoteric surfactants are imidazolines and betaines, e.g., lauric and myristic imidazolines and betaines, and amidopropylbetaines.

The compositions of the invention may include “sperm activation” agents. “Sperm activation agents” refers to the process of converting non-motile, functionally inactive sperm to a sperm in a state that is capable of fertilization. Examples of sperm activation agents include, but are not limited to, ions (such as bicarbonate, sodium, calcium, magnesium and manganese), salts, hyaluronidase (such as PH-20), albumin, high-density lipoprotein, progesterone, peptides, nucleosides, nucleotides, cyclic AMP, small molecules, proteins, antibodies, chemokine, cytokines, prostaglandins, caffeine, aspirin, carbon monoxide, carbon dioxide, nitric oxide, a combination thereof or other agents that are chemically, functionally, or physiologically equivalent or similar. As another example, the sperm are stored in a functionally inactive state in the cauda epididymis in an environment that contains high concentration of potassium ions, low sodium ions and very low bicarbonate ions. However, the composition of capacitation-inducing oviductal fluid and media comprises high concentration of various ions (such as sodium, calcium and bicarbonate) and low concentrations of others (such as potassium).

The compositions of the invention may include “function enhancing” agents. “Function enhancing” agents may include, but are not limited to, water-based solutions that are non-spermicidal, sperm friendly, egg friendly and facilitate fertilization. The function enhancing agents may also include, but are not limited to, compositions that maintain the viability of sperm and other cells, sperm and other cell energy-boosting agents, antioxidants and oxygen scavengers, fertilization facilitation agents, embryo attachment facilitation agents and/or other physiologic function enhancing agents. Function enhancing agents would greatly improve the chances of sperm-egg binding, thereby improving the fertilization potential, among other things. Similarly, use of novel compositions containing viability maintaining agents or fertilization and other function enhancing agents in sperm wash and sperm isolation media would also improve the chances of sperm-egg binding, thereby improving the fertilization potential, among other things.

The compositions of the invention may additionally include acids, such as, but not limited to, citric acid or lactic acid, among other acids.

The compositions of this invention may also include local analgesics and/or non-steroidal anti-inflammatory agents. The analgesics and non-steroidal anti-inflammatory agents may preferably include, but are not limited to, aspirin, inbuprofen, indomethacin, phenylbutazone, bromfenac, fenamate, sulindac, nabumetone, ketorolac, and naproxen and the like. The compositions may additionally include steroids such as corticosteroids and hydrocortisone acetate.

The compositions of this invention may also include one or more spermicides. Suitably, the spermicides may include, but are not limited to, nonoxynol-9, octooxynol-9, dodecaethyleneglycol monolaurate, Laureth 10S, and Methoxypolyoxyethyleneglycol 550 laureate and the like.

In another embodiment, the compositions of this invention may include polymers such as organosilicon compounds to act as a lubrication. Suitably, the organosilicon compound may include, but is not limited to, dimethicone (polydimethylsiloxane) or cyclopentasiloxane.

Another embodiment of this invention may include the use of compositions for vulvovaginal use containing one or more hormones, such as for treating a decrease in estrogen secretion in the woman in need of estrogen replacement, or in women with vaginal atrophy. Suitably, the hormones may include, but are not limited to, estrogen selected from the group consisting of estradiol, estradiol benzoate, estradiol cypionate, estradiol dipropionate, estradiol enanthate, conjugated estrogen, estriol, estrone, estrone sulfate, ethinyl estradiol, estrofurate, quinestrol and mestranol. Hormones such as prostaglandin and testosterone may also be used.

Additional medicaments that may be included in the compositions of the invention may include, but are not limited to, vasodilators, calcium channel blocks, cholinergic modulators, alpha-adrenergic receptor antagonist, beta-adrenergic receptor agonists, camp-dependent protein kinase activators, superoxide scavengers, potassium channel activators, estrogen-like compounds, testosterone-like compounds, benzodiazepines, adrenergic nerve inhibitors, HMG-CoA reductase inhibitors, smooth muscle relaxants, adenosine receptor modulators and adenylyl cyclase activators. Such agents include phosphodiesterase-5 inhibitors and the like.

In another embodiment, the compositions of this invention may be used to treat different aspects of female sexual dysfunction such as female sexual arousal disorder, hypoactive sexual desire disorder, orgasmic disorder and the like) as well as those that treat dyspareunia and/or vaginis-mus, or vulvodynia and to relieve pain upon intercourse.

### EXAMPLES

### Example 1

The compositions of the invention are combined together at a temperature of 40 to 80 degrees Celsius. The components are added together with an ingredient % of glycerin at 90, and 1,3-propanediol at 10. The final mixture has anti-microbial properties, as indicated by the results of a microbial test challenge (see Table 1):

TABLE 1

| | S. | | P. | | |
|---|---|---|---|---|---|
| Test Interval | auresus | E.coli | aeruginosa | C.albicans | A.niger |
| Initial | 2.0 × 10⁵ | 1.9 × 10⁵ | 1.1 × 10⁵ | 2.9 × 10⁵ | 1.1 × 10⁵ |
| Concentration | | | | | |
| (Inoculum/gm) | | | | | |
| Day 7 | 0 | 0 | 0 | 0 | 0 |
| Day 14 | 0 | 0 | 0 | 0 | 0 |
| Day 28 | 0 | 0 | 0 | 0 | 0 |

### Example 2

The compositions of the invention are combined together at a temperature of 40 to 80 degrees Celsius. The components are added together with an ingredient % of glycerin at 76, and 1,3-propanediol at 24.

### Example 3

The compositions of the invention are combined together at a temperature of 40 to 80 degrees Celsius. The components are added together with an ingredient % of glycerin at 70, and 1,3-propanediol at 30.

What is claimed is:

1. A personal care composition comprising:
a. glycerin, and
b. 1,3-propanediol.

2. The composition of claim 1, wherein the glycerin is present in an amount of about 5% to about 95% by weight.

3. The composition of claim 1, wherein the 1,3-propanediol is present in an amount of about 5% to about 95% by weight.

4. The composition of claim 1, wherein the composition is substantially free of preservatives.

5. The composition of claim 1, further comprising at least one additive.

6. The composition of claim 5, wherein the at least one additive is selected from the group consisting of vitamins, honey, alkylene glycol, surfactants, polymers, oligomers, sugars, essential oils, petrolatum, mineral oils, dimethicones, cyclopentasiolxane, flavoring agents, fragrances, aromas, medicinals, colorants, and extracts.

7. The composition of claim 1, wherein the composition is a warming lubricant.

8. The composition of claim 1, further comprising a pharmaceutical.

9. The composition of claim 8, wherein the pharmaceutical may be used to treat female sexual dysfunction.

10. A method of using the composition of claim 1 comprising applying the composition to a mucosal membrane.

11. The method of claim 10, wherein the membrane may be of the vulva, vagina, mouth, or skin.
